(19) United States
(12) Patent Application Publication
(10) Pub. No.: US 2003/0049312 A1
(43) Pub. Date: Mar. 13, 2003

(54) Hard gelatine capsules containing sustained-release plant extracts and method for production thereof

(76) Inventors: Frauke Gaedcke, Waldesch (DE); Willi Berkulin, Alsbach (DE)

Correspondence Address: JACOBSON HOLMAN PLLC, 400 SEVENTH STREET N.W., SUITE 600, WASHINGTON, DC, 20004, US

(21) Appl. No.: 10/221,312

(22) PCT Filed: Nov. 30, 2000
(86) PCT No.: PCT/EP00/11987

(51) Int. Cl. A61K 35/78; A61K 9/64; B29C 47/88

(52) U.S. Cl. 424/456; 424/725; 264/211.12

(57) ABSTRACT

The hard gelatin capsules contain sustained-release plant extracts containing a free-flowing filling consisting of compactates of the plant extracts coated with film agents or of mixtures of the plant extracts with auxiliary agents. They are prepared by subjecting the plant extracts, which are undiluted or mixed with auxiliary agents, to pressing to ribbons, mechanically comminuting and freeing from the oversize and undersize, coating with film-forming agents and filling into hard gelatin capsules.

The present invention relates to hard gelatin capsules containing sustained-release plant extracts and a method for production thereof.

According to the prior art, hard gelatin capsules comprising sustained-release active substances are prepared by processing the active substance together with release-sustaining agents, especially fats, waxes or alginates, in a fluidized bed granulator to give pellets. This preparation method is very complex and thus very cost-intensive. Plant extract are frequently sensitive to water and temperature, so that it is often hardly possible to prepare useful sustained-release pellets which are suitable for capsule fillings.

It is the object of the present invention to provide hard gelatin capsules containing sustained-release plant extracts which can be prepared safely, simply and with low costs and at the same time ensure that the plant extracts are not damaged by water and temperature.

According to the invention, this object is achieved by the hard gelatin capsules containing a free-flowing filling of compactates of the plant extracts coated with film agents or of mixtures of the plant extracts with auxiliary agents.

The preparation of the hard gelatin capsules is effected by

a) pressing the plant extracts, which are undiluted or mixed with auxiliary agents, to ribbons;

b) mechanically comminuting the ribbons;

c) separating the oversize and undersize from the comminuted ribbons;

d) coating the desired medium fraction with film-forming agents; and

e) filling into hard gelatin capsules.

If the plant extracts cannot be employed undiluted, they may be previously mixed with auxiliary agents, which also facilitate compacting to ribbons in general. Typical and preferred auxiliary agents are lactose, sucrose, calcium carbonate, polyethylene glycols, calcium hydrogenphosphate, cellactose, microcrystalline cellulose, cellulose derivatives, cross-carmellose and mixtures thereof.

The grain size distribution of the compactates coated with film agents is generally between 0.2 and 1.5 mm, preferably from 0.3 to 1.25 mm. Such compactates coated with film-forming agents are free-flowing and therefore can be filled into hard gelatin capsules without difficulties. Both the compacting and the coating with film-forming agents cause the required sustained release upon administration.

The desired grain-size distribution can be achieved most simply by passing through a first coarser sieve, the mesh size determining the grain size upper limit, and vibrating on a second, narrow-meshed sieve, the mesh size now determining the grain size lower limit.

However, it is altogether possible as well to separate this desired medium fraction from the initially comminuted mixture of the different grain sizes by air classification.

Said compacting, comminuting and separating of the desired medium grain size fractions can be effected, for example, in roll presses followed by a downstream milling system, followed by a downstream sieving machine. Such machines are supplied, for example, by Gerteis Maschinen+Processengineering AG under the designation of GMP-Polygran Walzenpresse.

In such devices, the undiluted, powdery plant extract or its mixture with auxiliary agents is metered from a silo, at the same time effecting a preliminary deaeration, The press rolls produce ribbons, which are broken, for example, by oscillating granulators and passed through a sieve to form the desired grain size. The oversize may optionally be recycled to the comminuting stage, and the undersize may be fed to the still powdery starting material if desired.

As the film-forming agents, there may be used the film-forming agents customary in the pharmaceutical industry, such as zein, shellac or the various Eudragit® solutions. Especially from the various Eudragit® solutions, coatings having a more or less pronounced release-sustaining effect can be prepared, so that the overall release-sustaining effect of the hard gelatin capsules can be adjusted.

As the plant extracts which are to be filled in sustained-release hard gelatin capsules, virtually all plant extracts may be used which are too quickly acting in a non-sustained form and which are not released sufficiently slowly. Examples thereof include Saint-John's wort, Echinacea, valerian and garlic.

1. Hard gelatin capsules containing sustained-release plant extracts, characterized by a free-flowing filling consisting of compactates of the plant extracts coated with film agents or of mixtures of the plant extracts with auxiliary agents.

2. The hard gelatin capsules according to claim 1, characterized in that said auxiliary agents are lactose, sucrose, calcium carbonate, polyethylene glycols, calcium hydrogenphosphate, cellactose, microcrystalline cellulose, cellulose derivatives, cross-carmellose or mixtures thereof.

3. The hard gelatin capsules according to claim 1 or 2, characterized in that said compactates coated with film agents have a grain size distribution of from 0.2 to 1.5 mm, preferably from 0.3 to 1.25 mm.

4. The hard gelatin capsules according to any of claims 1 to 3, characterized in that the film-forming agents are zein, shellac or Eudragit® solutions.

5. A method for the preparation of the hard gelatin capsules with sustained-release plant extracts, characterized in that
a) the plant extracts, which are undiluted or mixed with auxiliary agents, are pressed to ribbons;
b) the ribbons are mechanically comminuted;
c) the oversize and undersize are separated from the comminuted ribbons;
d) the desired medium fraction is coated with film-forming agents; and
e) filled into hard gelatin capsules.

6. The method according to claim 5, characterized in that lactose, sucrose, calcium carbonate, polyethylene glycols, calcium hydrogenphosphate, cellactose, microcrystalline cellulose, cellulose derivatives, cross-carmellose or mixtures thereof are used as auxiliary agents.

7. The method according to claim 5 or 6, characterized in that steps b) and c) are performed by passing through a first coarser sieve, the mesh size determining the grain size upper limit, and vibrating on a second, narrow-meshed sieve, the mesh size now determining the grain size lower limit.

8. The method according to claim 5 or 6, characterized in that steps b) and c) are performed by air classification.

9. The method according to any of claims 5 to 8, characterized in that the grain size distribution of said medium fraction ranges from 0.2 to 1.5 mm, preferably from 0.3 to 1.25 mm.

10. The method according to any of claims 5 to 9, characterized in that zein, shellac or Eudragit® solutions are used as the film-forming agents.
